(19) United States
(12) Patent Application Publication
(10) Pub. No.: US 2004/0122451 A1
(43) Pub. Date: Jun. 24, 2004

(54) Method and device for closing and fusing suture loops

(76) Inventor: Timothy J. Wood, Wilmington, MA (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/328,854

(22) Filed: Dec. 23, 2002

(51) Int. Cl. A61B 17/04

(52) U.S. Cl. 606/148

(57) ABSTRACT

A suture fusing device comprises a shaft having an energy transducer at its distal end. Sutures received through an aperture in the transducer, and the device may be used to apply tension to a suture loop to close a tissue puncture or wound. After the puncture wound has been closed, energy can be applied through the transducer to fuse opposed strands of the suture together.

### FIELD OF THE INVENTION

The present invention relates generally to surgical methods and devices. More particularly, the present invention relates to a method and device for closing a suture loop over a tissue puncture or penetration by first applying tension to close the loop and thereafter applying energy to fuse opposed strands of the loop together.

### BACKGROUND

The use of suture for closing incisions and wounds is a preferred technique of surgeons and many other physicians. While a variety of other techniques are now available, such as stapling, the use of “tissue glues,” and the use of collagen for closing vascular punctures, the use of suture is often preferred since it provides a reliable and tight closure for any incision or wound where the suture can be properly placed, tightened, and secured.

While suturing is relatively straight forward in most open surgical procedures, the placement and tying of sutures in laparoscopic and other minimally invasive procedures can be problematic. In order to provide for suturing under such circumstances, a variety of devices have been developed for the remote placement and tying of sutures through canulas under video observation. Frequently, a sliding knot will be formed in the suture, and it will be necessary to use a tool, such as a knot pusher, for cinching the slidable knot over the loop.

Methods and devices have been proposed for closing vascular punctures using suture loops passed through opposed edges of a puncture site. In one exemplary use, such methods and devices are used to close punctures in the femoral artery of a patient who has undergone a catherization procedure to treat a cardiovascular condition.

In one exemplary method, a suture-applying device is introduced through a tissue tract and uses one or more needles to pass suture through the blood vessel wall on opposite sides of the penetration. The suture is then drawn upward through the tissue tract, and the suture tightened and secured to close the wound, typically by forming a knot just over the adventitial surface of the blood vessel wall.

The ightening and securing of suture loops in both vascular and alaparoscopic prodedures is problematic due to the limited access. In the case of vascular procedures, the know or other securing means must be formed and placed directly over the adventitial surface in order to securely close the blood vessel wall puncture. This means that the suture must be tied or otherwise manipulated a the distal end of the tissue tract which is typically at least 2 cm in length for common femoral penetrations. In laparoscopic procedures, the suture must be secured through a narrow diameter trocar, typically having a diameter of at most 10 mm, and often oly 5 mm. The nee to perform sequential and/or multiple tightening and securing steps for the suture loops in both vascular and laparoscopic procedures is disadvantageous.

For these reasons, it would be desirable to provide improved methods and devices for tightening and securing suture loops in vascular, laparoscopic, and other restricted access surgical procedures. It would be particularly desirable if such methods and devices would allow both tightening of the suture loop, and subsequent securing of the strands of the suture loop, and subsequent securing of the strands of the suture loop together, in a minimum number of steps. Most preferably, the methods and devices of the present application would provide for tightening and securing of opposed strands of the suture loop in a procedure where the opposed strands ar first pulled to tighten the loop and thereafter secured together in a single step.

### SUMMARY

According to the present invention, opposed strands of a suture loop are secured together by advancing an aperture along the strands, usually in a direction normal to the wound or other tissue puncture which is being closed. After the aperture has been advanced to a point where sufficient tension is applied on the sutures to close the wound, energy is applied through the aperture to fuse the strands of the suture together. Optionally, a radially inward pressure can be applied to the suture as the energy is being applied, for example by selectively collapsing or constricting the aperture over the suture. The method of the present invention thus provides f or both closing of the loop and subsequent securing of the strands together to secure the loop in an efficient manner which, unlike many prior art methods and devices, requires no prior tying or twisting of the suture strands.

The present invention also provides a device for securing the opposed strands according to the above-described method. The device comprises a shaft having a proximal end, a distal end, and a suture-receiving path therebetween, typically in the form of a closed lumen or open axial channel. An energy transducer is disposed at the distal end of the shaft and has a suture-receiving aperture aligned with the suture-receiving path in the shaft. The method can thus be performed by drawing the opposed strands of the suture through the aperture and the suture-receiving path to first close the suture loop over the wound or tissue puncture. Energy may then be applied through the energy transducer to fuse the strands together.

In specific aspects of the present invention, the energy may be heat, ultrasonic, or any other form of energy which may be conveniently applied to melt and fuse the suture together. The aperture within the device may be a closed orifice or an open slot, an aperture may be further modified to provide for closing over the suture in order to apply pressure to enhance fusing when the energy is applied. The shaft preferably has a narrow diameter, typically below 10 mm, and preferably below 5 mm, and a sufficient length, typically greater than 10 cm, and preferably greater than 15 cm, in order to permit use in vascular, laparoscopic, and other minimally invasive procedures where access to the wound or puncture site to be closed is limited.

The methods and devices of the present invention are particularly useful for closing punctures in blood vessel walls which result from transluminal procedures, such as angiography, angioplasty, atherectomy, and the like. The methods and devices will also be particularly useful in laparoscopic and other minimally invasive procedures where the device will be introduced through a trocar into a body cavity to close a puncture or wound site therein.

### BRIEF DESCRIPTION OF THE DRAWINGS

FIG. 1 is a perspective view of a suture fusing device constructed in accordance with the principles of the present invention.

FIG. 2 is a side, partial cross-sectional view of the device of FIG. 1.

FIG. 3A is a side view of an alternate embodiment of the suture fusing device of the present invention having an axial slot for receiving the suture to be fused.

FIG. 3B is a detailed view of the distal end of the device of FIG. 3A.

FIG. 4A illustrates a second alternate embodiment of the suture fusing device of the present invention having a slot formed in a distal transducer, wherein the suture is brought back on the outside of the device shaft.

FIG. 4B is a detailed view of the distal end of the device of FIG. 4A.

FIGS. 5A-5C show one embodiment of the distal and of the device having a suture engaging mechanism.

FIG. 6 is a side view of an alternate embodiment of a suture fusing device of the present invention having a suture-tensioning mechanism.

FIGS. 7A-7D illustrate use of the suture fusing device of FIGS. 1 and 2 for closing and securing a suture loop formed through a vascular penetration located at the distal end of the tissue tract.

### DETAILED DESCRIPTION

Devices according to the present invention may include a shaft having a proximal end, a distal end, and an energy transducer secured to the distal end. The transducer will have a suture-receiving aperture which is aligned with the axial direction of the shaft. The aperture will be able to receive the opposed suture strands in order to tighten the suture loop by advancing the aperture over the strands. After proper tension is applied to the strands to close the suture loop, energy can be applied to the suture through the transducer to secure the opposed strands together.

The shaft of the device will generally be rigid, having a length in the range from about 5 cm to about 15 cm, preferably from about 7 cm to about 13 cm. In order to permit introduction to remote target locations, the width or diameter of the shaft should not exceed about 10 mm at any point along its length (i.e. the length which is to be introduced through the canula or tissue tract to the remote suturing location). Optionally, the shaft may include a suture-receiving path, such as a lumen or axial channel for guiding the suture from the aperture to the proximal end of the shaft.

The energy transducer can be an electrical resistance heater, an ultrasonic transducer, or the like, which is capable of delivering sufficient energy to fuse the opposed suture strands together. The transducer will be connected to an appropriate power source, e.g. an alternating or direct current power supply in the case of a resistance heating element or an ultrasonic power supply in the case of an ultrasonic transducer. Such power sources are commercially available. Typically, the transducers will be connected to the power source through an electrical cord which extends from a proximal end of the device. Alternatively, a battery can be provided as a power source. Optionally, a switch could be provided within the suture fusing device in order to selectively apply energy to the transducer. Alternately, the user could rely on a switch on the power source itself, or on an associated foot switch or other remote switch for the power source.

The suture fusing methods and devices of the present invention are intended for closing, tightening, and securing suture loops which have been previously formed in wounds or other tissue penetrations which are desired to be sutured closed. While the suture loops may be formed in a conventional manner by a wide variety of known surgical procedures, including open surgical procedures, the methods and devices of the present invention are particularly useful f or securing remotely located suture loops, particularly those formed through vascular penetrations located at the distal end of the tissue tract or within a remote body cavity accessed through a canula in laparoscopic or other minimally invasive surgical procedures.

The methods and devices of the present invention rely on applying energy, typically heat or ultrasonic energy, to fuse opposed strands of suture together in place of tying. Thus, the suture used in the procedures of the present invention will be capable of being melted or otherwise fused together, typically being composed of a synthetic polymer, such as a polyamide polymer (e.g. a nylon), or a polyester polymer (e.g. Dacron®). Many commercially available sutures are fusible, such as DEKNATEL monofilamental nylon, ETHICON, and ASHAWAY.

The methods and devices of the present invention will generally avoid the need to pre-twist or pre-tie the suture prior to fusing. Previous suture fusing devices have generally relied on fusing a suture which has been previously twisted in order to provide a desired tensioning prior to fusing. With the present application, in contrast, the suture strands may be left generally parallel to each other, without any pre-twisting or pre-tying, with tensioning and closing of the suture loop being provided by the device itself. After the proper tensioning has been applied, the device can be used to fuse the opposed strands of suture together. Of course, the devices of the present invention could optionally be used with suture which has been pre-twisted or pre-tied, but this will not generally be necessary.

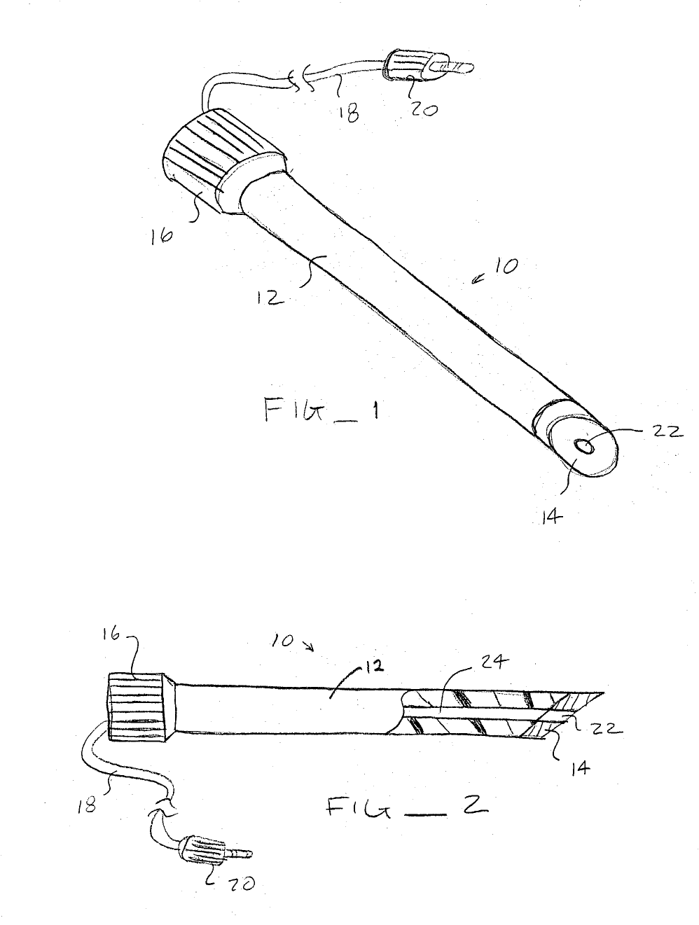

Referring now to FIG. 1, a first embodiment of a suture fusing device 10 constructed in accordance with the principles of the present invention will be described. The device 10 comprises a shaft 12 having a transducer 14 at its distal end and a hub 16 at its proximal end. The transducer 14 is an electrical heating element and may be connected to a power supply (not shown) by connecting cord 18 having an appropriate plug 20 at its free end. Alternatively, the power supply may be a battery (not shown) contained in a handle-like structure (not shown) or in the shaft 12 or hub 16.

The transducer 14 includes a central aperture 22 which is in the form of a closed orifice. As shown in FIG. 2, a central lumen 24 in the shaft 12 is axially aligned with the aperture 22 and extends proximally all the way through the proximal hub 16. Thus, as described in more detail below, suture may be drawn into aperture 22 and outward through lumen 24 as the suture fusing device 10 is used.

The transducer 14 is disposed at an oblique angle relative to the axial direction of shaft 12, typically being from about 30° to about 60°, and shown in FIG. 2 as about 45°. The inclined distal surface of the transducer 22 permits the transducer to be placed closely over a vascular penetration lying at the distal end of a tissue tract. As shown in greater detail below, such tissue tracts are normally disposed at an angle relative to the adventitial surface of the blood vessel wall. The inclined surface of the transducer 14 can offset this angle.

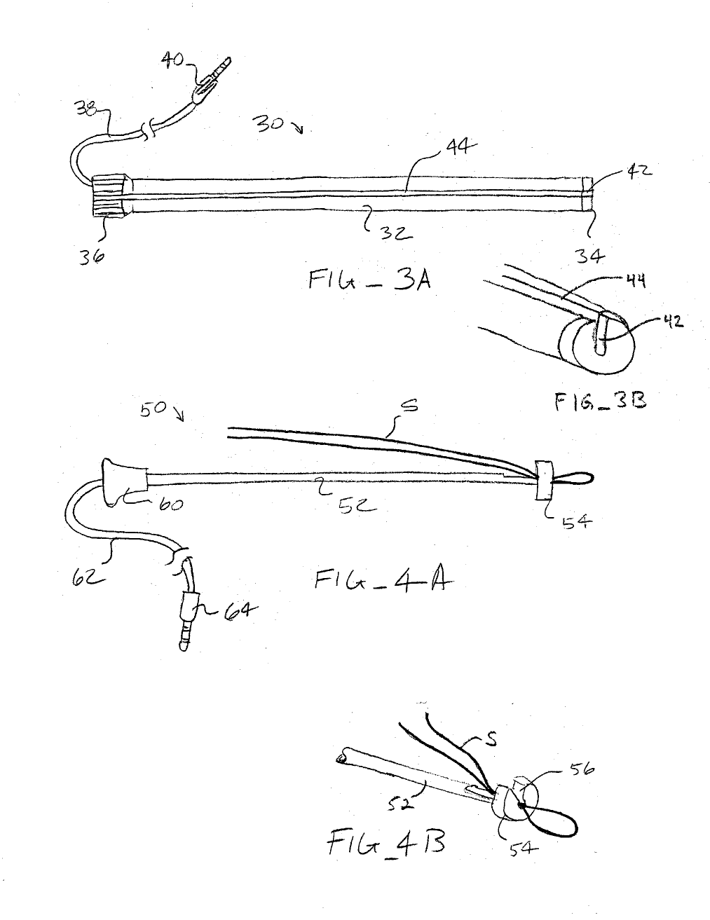

Referring now to FIGS. 3A and 3B, an alternate embodiment of the suture fusing device will be described. The device 30 includes a shaft 32, a transducer 34 at the distal end of the shaft, a hub 36 at the proximal end of the shaft, a power connection cord 38, and a plug 40. The device 30 is generally the same as device 10, except that a slot 42 is formed in the transducer 34 rather than the orifice 22 of device 10. Similarly, an axial channel 44 is formed in the shaft 32 instead of the closed lumen 24. Provision of the open slot 42 and open axial channel 44 facilitates introduction of the free ends of the suture into the device. That is, the suture strands can be simply laid into the slot 42 and channel 44 prior to use of the device, rather than having to thread the suture strands as required by device 10.

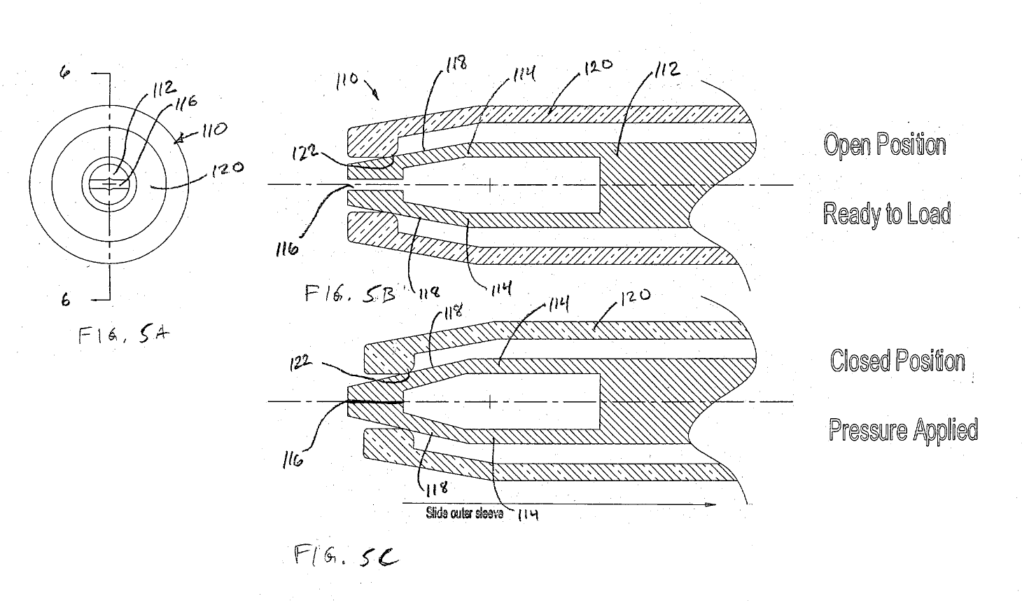

A second alternate embodiment 50 of the suture fusing device of the present invention is illustrated in FIG. 4A. The device 50 includes a shaft 52 having transducer 54 at its distal end. A proximal hub 60, connecting cord 62, and plug 64, will also be provided in a manner similar to the previous embodiments. The shaft 52 will have a smaller width or diameter relative to transducer 54 than the shafts 12 and 32 of devices 10 and 30 have relative to their respective transducers 14 and 34, respectively. Shaft 52 is not intended to receive a suture therethrough. As shown in FIG. 4B, suture S will pass through slot 56 in the transducer 54 and then proximally in parallel to the shaft 52. The slot 56 in transducer 54 will preferably have a V-shaped cross section in order to facilitate capture of the suture.

Additional features may be provided in various embodiments of the suture fusing devices of the present invention. For example, in some cases, it may be desirable to provide a mechanism for applying pressure to the suture while the energy is being delivered by the transducer. In another example, the transducer aperture may be closed over the suture while the energy is being applied. Alternatively, an anvil or other static member may be supplied for engaging the suture and pressing it against the transducer while energy is being supplied.

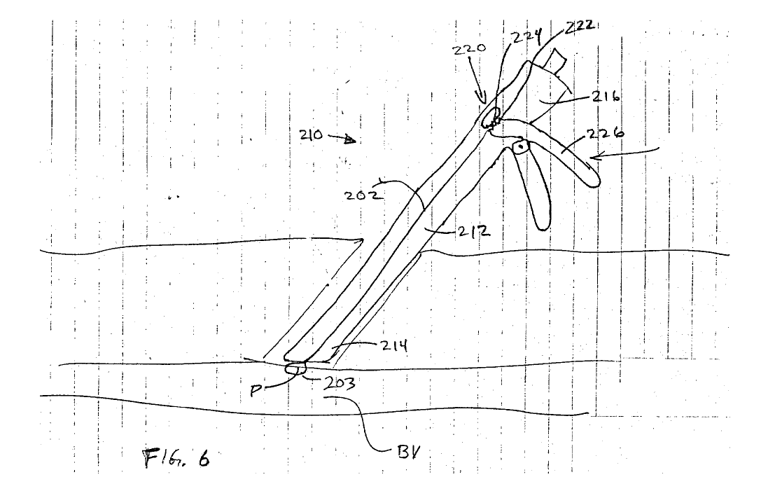

FIGS. 5A through 5B show an embodiment of a suture engaging mechanism 110 that can be provided on the distal end of the shaft of the device. FIG. 5A is an end view of the distal end of the device in which is shown the suture holding member 112 defining a portion of the suture receiving path 116. An outer sleeve 120 surrounds and can be coaxial with the suture-holding member 112.

FIG. 5B shows the suture engaging mechanism 110 in an open position in which the suture (not shown) may be loaded through the suture-receiving path 116. The suture holding member 112 includes two opposing, and distally extending arms 114, between which is defined a portion of the suture-receiving path 116. The outer surfaces of the arms 114 define tapered end surfaces 118 that taper toward the distal end of the suture-holding member 112. The arms 114 are bendable to constrict the suture-receiving path 116 as shown in FIG. 5C.

The suture engaging mechanism 110 further includes an outer sleeve 120 surrounding the arms 114 (as shown in FIG. 5A). The outer sleeve 120 is movable relative to the arms 114. The outer sleeve 120 has a bearing surface 122 that is engageable with the tapered surfaces 118 of the arms 114 as the outer sleeve is moved relative to the arms. As shown in FIG. 5C, the bearing surface 122 is engaged with the tapered surfaces 118 of the arms 114 such that the arms are bent to constrict the suture receiving path 116 as the bearing surface 122 (having a static inner diameter) is moved proximally along the increasing diameter of the tapered surfaces 118. In the embodiment shown in FIGS. 5A-5C, when suture strands (not shown) are received in the suture receiving path 116, the suture strands are held between the arms 114 when the outer sleeve is moved proximally toward the position shown in FIG. 5C.

Alternatively, the outer sleeve may be configured to include inner threads that mate with a threaded surface of the suture holding member 112 such that the outer sleeve is moved by being rotated. The rotation of the outer sleeve with respect to the suture-holding member would threadably advance the sleeve proximally (or advance the suture holding member distally) to bend the arms 114 to constrict the suture-receiving path 116.

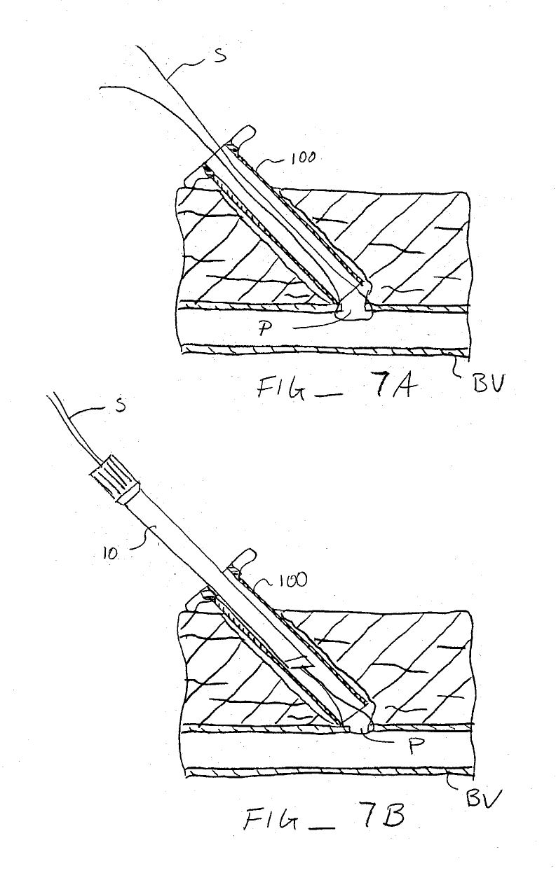

FIG. 6 shows an alternate embodiment of the invention in which a suture-fusing device 210 includes a suture tensioning mechanism 220. Tension is applied to the suture strands 202 in order to tighten the suture loop 203 around the puncture P in the blood vessel BV in this example of an application of the invention. The distal end 214 of the shaft 212 would include a transducer to apply energy to the suture strands to fuse the strands together, as described previously with reference to the various embodiments of the invention.

As shown in FIG. 6, device 210 has a shaft 212 having a distal end 214 and a proximal end 216. The suture tension mechanism 220 is preferably at or near the proximal end 216. The suture tension mechanism 220 includes a first suture grabbing surface 222 and a second suture-grabbing surface 224. The first suture grabbing surface 222, in the embodiment shown in FIG. 6, is movable to apply tension to the suture strands 202 when the suture strands are grabbed.

The suture tensioning mechanism 220 may further include a pivotable handle 226 that has the first suture grabbing surface 222 on its pivoted end. The pivotable handle 226 can be mounted to the shaft 212 at a pivot point such that distal movement of the handle 226 relative to the shaft moves the first suture-grabbing surface 222 proximally. Alternatively, the mechanism by which the first suture grabbing surface 222 is moved can be a sliding button or other mechanism known in the art to impart motion.

In the embodiment shown in FIG. 6, the suture tensioning mechanism 220 includes a second suture-grabbing surface 224. Movement of the pivotable handle 226 biases the first suture grabbing surface 222 against the second suture grabbing surface 224. At least the first suture-grabbing surface 222 moves proximally during the biasing motion. Thus, when suture strands are disposed between the suture grabbing surfaces 222 and 224, the strands are grabbed and tensioned in the proximal direction.

It should be noted that the second suture-grabbing surface 224 could be movable in the longitudinal direction with respect to the shaft 212. For example, the second suture grabbing surface 224 can be slidable or can be on a rotating body such that when the first suture grabbing surface 222 is biased against it, the surfaces can be moved proximally together to tension suture strands that are disposed therebetween. In an alternate embodiment, the suture tension mechanism can rotate a suture-grabbing surface to wind the suture strands to apply tension to the suture strands.

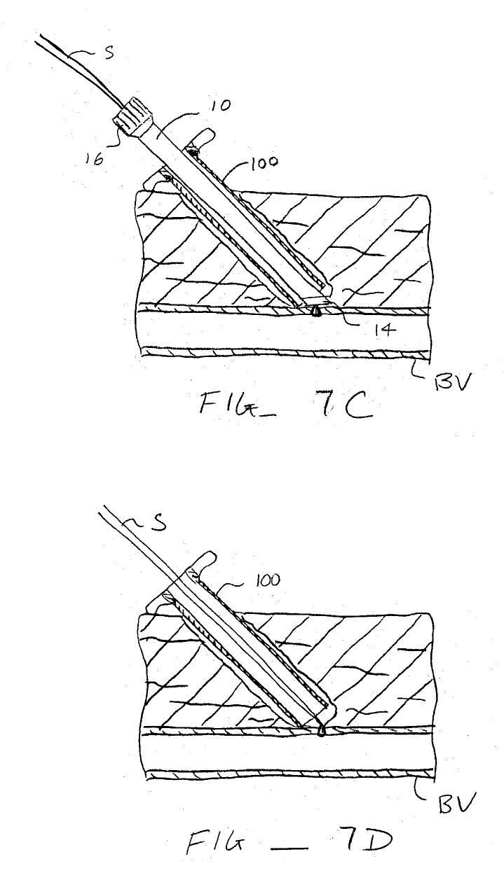

Referring now to FIGS. 7A-7D, use of the suture fusing device 10 for closing and securing a suture loop S located over a puncture P in a blood vessel BV will be described. After the suture has been passed through the blood vessel wall, the suture will extend outward through an access sheath 100, as illustrated, and it is necessary to apply sufficient tension to the suture to close the penetration P and thereafter fuse the opposed strands of the suture together.

The suture fusing device 10 is introduced over the opposed strands of suture S, as illustrated in FIG. 7B. Note that a single loop of suture is illustrated in FIGS. 7A-7D. Alternatively, a pair of suture loops having four opposed strands extending outward through the tissue tract will have been placed prior to closing of the puncture P and fusing of the suture.

As illustrated in FIG. 7C, the suture fusing device 10 is advanced fully through the access sheath 100 so that the transducer 14 lies immediately over the adventitial surf ace of the blood vessel wall. Sufficient tension is applied on the suture S to close the loop through the blood vessel wall. The tension will typically be applied manually, where the physician holds the hub 16 in one hand and pulls on the suture S extending proximally from the hub with the other hand. After sufficient tension is applied to fully closed puncture P, energy is applied through the transducer 14 to fuse the opposed strands of suture together. The device 10 can then be withdrawn, leaving the fused suture loop, as illustrated in FIG. 7D. The suture can then be trimmed, and the access sheath 100 removed.

Although the present invention has been described in connection with the preferred form of practicing it and modifications thereto, those of ordinary skill in the art will understand that many other modifications can be made thereto within the scope of the claims that follow. Accordingly, it is not intended that the scope of the invention in any way be limited by the above description, but instead be determined entirely by reference to the claims that follow.

What is claimed is:

1. A method for securing opposed strands of a suture loop, said method comprising:
advancing an aperture along said opposed strands to close the suture loop; and
applying energy the aperture to fuse said strands together.

2. The method of claim 1, further comprising applying pressure to the suture strands while applying energy to enhance fusing.

3. A method as in claim 1, wherein said aperture is advanced by holding the opposed suture strands and pushing the aperture distally over said strands.

4. A method as in claim 1, wherein the energy applying step comprises applying at least one of heat energy and ultrasonic energy.

5. A method as in claim 1, wherein the aperture is advanced through a tissue tract to close a suture loop over a blood vessel puncture at a distal end of the tissue tract.

6. A method as in claim 1, wherein the aperture is advanced through a trocar lumen to close a suture loop in a body cavity.

7. A method for securing opposed strands of a suture loop, said method comprising:
providing a fusing device including an elongate shaft having a suture-receiving aperture at a distal end thereof;
drawing the opposed strands of suture through the aperture and axially along the shaft to close the suture loop; and
applying energy through the aperture to fuse said strands together.

8. A method as in claim 7, wherein the drawing step comprises pulling the suture strands through an axial lumen or channel in the shaft.

9. A method as in claim 8, wherein the aperture is an open slot and the suture is laid into the slot and channel before drawing.

10. A method as in claim 8, wherein the aperture is a closed orifice and the suture is threaded through the orifice and lumen before drawing.

11. A method as in claim 8, wherein the providing step comprises providing a fusing device with means for closing the aperture, and the method further comprises applying pressure to the suture by closing the aperture while applying energy to enhance fusion.

12. A method as in claim 7, wherein the opposed strands are drawn by holding the strands in one hand and advancing the fusing device in the other hand until sufficient tension has been applied to close the suture loop with a desired tightness.

13. A method as in claim 7, wherein the suture strands are drawn through a tissue tract to close a suture loop over a blood vessel puncture at a distal end of a tissue tract.

14. A method as in claim 7, wherein the suture strands are drawn through a trocar to close a suture loop in a body cavity.

15. A device for fusing opposed strands of suture together, said device comprising:
a shaft having a proximal end, a distal end, and a suture-receiving path therebetween; and
an energy transducer at the distal end of the shaft, said transducer having a suture-receiving aperture aligned with the suture-receiving path in the shaft, wherein the opposed suture strands may be drawn through the aperture and suture-receiving path in the shaft and energy is applied through the transducer to fuse the strands together.

16. The device of claim 15, wherein the suture-receiving path comprises an axial lumen or channel in the shaft.

17. A device as in claim 15, wherein the energy transducer comprises an electrical resistance heater or ultrasonic transducer.

18. A device as in claim 15, wherein the suture-receiving aperture comprises a closed orifice or an open slot.

19. A device as in claim 15, further comprising means closing the suture-receiving aperture over the suture strands to apply pressure to said strands.

20. A device as in claim 15, wherein the shaft has a length in the range from about 7 cm to about 15 cm and a maximum width at any point along said length of about 10 mm.

21. The device of claim 15 further comprising a suture-engaging mechanism at the distal end of the shaft.

22. The device of claims 21 wherein the suture-engaging mechanism includes a suture holding member defining at least a portion of the suture-receiving path, the suture-holding member having arms bendable to constrict the suture-receiving path.

23. The device of claim 22, further comprising an outer sleeve surrounding the arms and movable relative to the arms, the arms having tapered end surfaces, the sleeve having a bearing surface engageable with the tapered surface as the outer sleeve is moved relative to the arms such that when the bearing surface is engaged with the tapered surface, the arms are bent to constrict the suture-receiving path.

24. The device of claim 15, further comprising a suture-tensioning mechanism at the proximal end of the shaft.

25. The device of claims 24, wherein the suture-tensioning mechanism includes a pivotable handle having a first suture-grabbing surface, the suture-tensioning mechanism including a second suture-grabbing surface, wherein the pivotable handle biases the first and second suture-grabbing surfaces toward each other and slightly proximally.

26. A device for fusing opposed stands of suture together, the device comprising:
a shaft having a proximal end, a distal end, and a suture-receiving path therebetween;
an energy transducer at the distal end of the shaft, said transducer having a suture-receiving aperture aligned with the suture-receiving path in the shaft, wherein the opposed suture strands may be drawn through the aperture and suture-receiving path in the shaft and energy is applied through the transducer to fuse the strands together; and
a suture-tensioning mechanism at the proximal end of the shaft having at least one suture-grabbing surface that is movable to apply tension to the suture strands when the suture strands are grabbed.

27. The device of claim 26, wherein the suture-tensioning mechanism includes first and second suture-grabbing surfaces, wherein the suture strands may be grabbed therebetween.

28. The device of claim 27 wherein the first and second suture-grabbing surfaces are movable proximally to tension the suture strands.

29. The device of claim 26, wherein the suture-tension mechanism rotates the suture-grabbing surface to wind the suture strand to apply tension to the suture strand.
